(19) United States
(12) Patent Application Publication
(10) Pub. No.: US 2004/0028556 A1
(43) Pub. Date: Feb. 12, 2004

(54) Method for the sterilisation of objects

(76) Inventors: Robert Frost, Landshut (DE); Peter Awakowicz, Muenchen (DE)

Correspondence Address: CROWELL & MORING LLP, INTELLECTUAL PROPERTY GROUP, P.O. BOX 14300, WASHINGTON, DC, 20044-4300, US

(21) Appl. No.: 10/363,546

(22) PCT Filed: Aug. 9, 2001
(86) PCT No.: PCT/EP01/09192

(30) Foreign Application Priority Data
Sep. 7, 2000 (DE) 100441173

(51) Int. Cl. A61L 2/20

(52) U.S. Cl. 422/33

(57) ABSTRACT

Described is a process for the chemical sterilization of objects, in particular of medical objects, which are packed in a non-sterile state into a bacteria-impermeable, but gas-permeable non-sterile package and subsequently sterilized using a diffusionable, reactive, gas-like medium. The medium is an overheated hydrogen peroxide vapor, which drops below dew point in the inside of the package and is thus forced to condensate. The condensate is subsequently returned to the vapor phase by evacuating the package, and thereafter pumped out.

### BACKGROUND AND SUMMARY OF THE INVENTION

The present invention relates to a process for sterilizing objects, in particular medical objects, which are packed in a non-sterile state into a bacteria-impermeable, but gas-permeable non-sterile package and subsequently, for the purposes of chemical sterilization, dampened by a reactive gas-like medium which penetrates into the package as a result of a difference in pressure, and which is replaced by a sterile gas after the sterilization process.

A process of this type is prior art in international patent application WO 96/39340. This type of process is applied, for example, to empty medical syringes, which, after sterilization, are supplied in a closed package to pharmaceutical companies for filling. It is hereby possible that the closed package is in turn to be found inside an additional closed package, which also has areas which are gas-permeable, but impervious to microorganisms. In this way, not only the objects to be sterilized can be kept permanently sterile, but also the packaging directly surrounding them. The extremely toxic gas ethylene oxide in its vapor phase is usually used as the reactive medium in this case. This results subsequently in a complicated process whereby the sterilized objects, including the inner surfaces of the packaging, must be cleaned completely of any ethylene oxide residue.

It is an object of the present invention to omit the use of the extremely toxic gas ethylene oxide in a process of the above mentioned type, and in addition to simplify as far as possible the removal of the reactive gas-like medium.

This object has been achieved in accordance with the present invention in that the medium used is overheated hydrogen peroxide vapor which drops below dew point in the inside of the package and is thus forced to condensate, whereby the condensate is subsequently returned to its vapor phase by means of evacuating the package and thereafter pumped out.

The basic principle of sterilizing objects by means of condensated hydrogen peroxide is known in European published patent 243 003; however, in the known process, the hydrogen peroxide residue is dried off after sterilization in a complicated process using heated air.

For the actual killing of the microorganisms, the hydrogen peroxide must be “activated”, whereby the word “activate” is undefined. A chemical and/or a physical change takes place brought about by the heat being applied to the hydrogen peroxide, which effects the destruction of the microorganisms. In the process according to the present invention, the “activating” of the hydrogen peroxide takes place exactly when it is needed for the sterilization process, that is during condensation. The overheated hydrogen peroxide vapor causes the surfaces to be sterilized to be covered by a thin, homogenous film of fluid. For this purpose, a microscopically thin condensation coating, which is almost invisible to the naked eye, is enough to ensure a sufficient sterilizing effect in the shortest possible time. Even in the case of condensation arising from relatively cold hydrogen peroxide vapor, that is, when the process is carried out in a vacuum, sufficient vapor energy is set free which results in the heating of the arising liquid phase. Because, however, of the very thin film of fluid, the temperature of the surfaces to be sterilized is hardly increased at all. Because the hydrogen peroxide vapor is always mixed with water vapor, and the boiling point of hydrogen peroxide is higher than that of water, the hydrogen peroxide condensates before the water does, so that at first an almost pure condensation layer of liquid hydrogen peroxide arises with a water layer disposed on top. This results, at exactly the right moment, to an increase in the concentration of hydrogen peroxide in the mixture. The inside of the package is exposed to a vacuum with a maximum pressure of no more than a few tens of millibars. The removal of the hydrogen peroxide no longer needs to be carried out by subsequent heating, but rather evaporation occurs by means of simple evacuation at a pressure below boiling point. The sterile gas, which subsequently replaces the suctioned off hydrogen peroxide, does not need to be sterile initially, as it becomes sterile in any case when it flows into the bacteria-impermeable package. The sterile gas can thus be normal air.

The process according to the present invention includes such variations in which a stable container containing the objects to be sterilized is in turn contained in a second container or in a bag, wherein the second container or the bag are also gas-permeable, but bacteria-impermeable, for example for syringes with injecting needles. The entire package can be sterilized in one operation.

For the purpose of the present invention, the overheated hydrogen peroxide vapor is fed from an evaporator into a sterilization chamber, which holds at least one package. It is, however, advantageous when a plurality of such packages are simultaneously sterilized in the sterilization chamber. It is hereby favorable to apply the principle of so-called continuous supersaturation. Overheated hydrogen peroxide vapor hereby flows out of the evaporator into the sterilization chamber due to the set drop in pressure, namely constantly over a certain period of time. Liquid hydrogen peroxide is continuously evaporated over a certain length of time, whereby the overpressure in the evaporator is maintained, which results in hydrogen peroxide vapor being continuously fed into the sterilization chamber. Because the temperature and thus the pressure in the evaporator is higher, the hydrogen peroxide vapor expands in the sterilization chamber, which in turn leads to a cooling-down and thus to a condensation process. In addition thereto, however, the pressure in the sterilization chamber is increased due to the constant flow of hydrogen peroxide vapor. Cooling down and pressure increase lead to a supersaturation of the vapor accumulating in the sterilization chamber, as both processes force the overheated hydrogen peroxide vapor below the dew point. As long as hydrogen peroxide vapor is fed in, condensation will occur on all accessible surfaces.

In an embodiment of the present invention, the condensation and the subsequent evacuation can be repeated at least once. The sterilizing effect is significantly improved by repeated vapor in-flow and pumping off at overall identical treatment times—in the region of ten minutes.

Alternatively it can be provided that condensation is repeated at least once, without an intermediary evacuation process. After the overheated hydrogen peroxide vapor has been fed into the package, there is initially a large increase in pressure. The condensation process which then sets in results in a loss of volume in the vapor phase, so that there is a significant drop again in pressure. The condensation surfaces then act as pumps, as the condensation can no longer generate pressure in the volume. The condensate is, in a way, “pumped out” of the volume. Thereafter, overheated hydrogen perodixe vapor can again flow in. This process can be repeated several times, whereby with each repetition of the flow in of hydrogen peroxide vapor, further amounts of vapor are brought into the package to be sterilized. Furthermore, because of the increase in pressure each time, the vapor volume located in the inside of the package can be forced just below the dew point, which then causes a further proportion of the vapor phase located therein to condensate.

The condensation can be accelerated or increased in that, after the hydrogen peroxide vapor has flowed in, the package is flooded with a sterile gas, which, for example, can be normal air, which is sterile in any case when it flows into the bacteria-impermeable package. Because the superheated hydrogen peroxide vapor located in the package and on the objects to be sterilized lies very close to the dew point, the pressure can be increased by means of the above mentioned flooding of, for example, the whole sterilization chamber. Due to this increase in pressure, the vapor phase still present is forced below the dew point and condensates further. The flooding process can in principle be repeated and also take place in the overpressure area. A further advantage of the increase in pressure by means of an auxiliary gas is that the condenstation of hydrogen peroxide vapor which has already been applied, is reinforced without the necessity for further application of a vapor. As a result, the amount of the medium to be subsequently pumped off can be reduced.

### BRIEF DESCRIPTION OF THE DRAWINGS

These and further objects, features and advantages of the present invention will become more readily apparent from the following detailed description thereof when taken in conjunction with the accompanying drawings wherein:

FIG. 1 is an installation for carrying out the process according to the present invention,

FIG. 2 is a multi-layered package for objects to be sterilized.

### DETAILED DESCRIPTION OF THE DRAWINGS

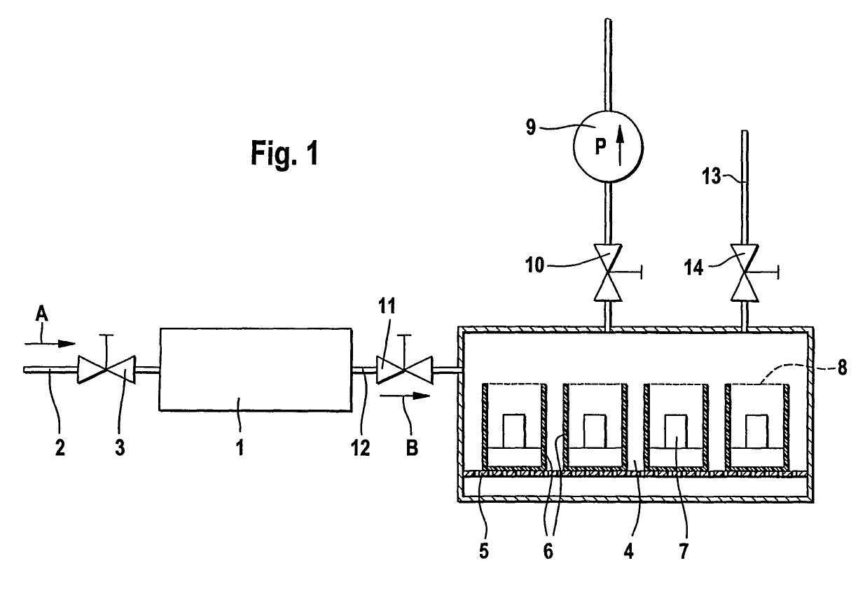

In the installation according to FIG. 1 a vapor mix consisting of overheated water steam and overheated hydrogen peroxide vapor is generated, whereby the design of the evaporator 1 is irrelevant. A watery solution of hydrogen peroxide at the desired degree of concentration of, for example 30% to 40%, is fed under pressure in direction A via a conduit 2 and a valve 3 to the evaporator 1.

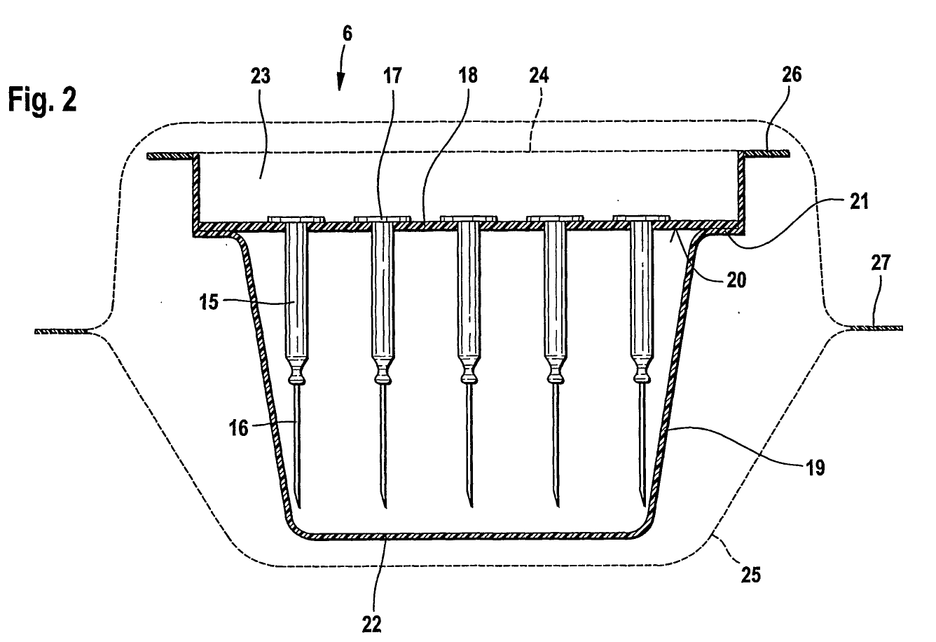

A sterilization chamber 4 is arranged downstream of the evaporator 1, in which sterilization chamber 4 packages 6, in which the objects 7 to be sterilized are packed, are located on a suitable supporting surface 5. Each package 6 is each closed by a lid 8, which is bacteria-impermeable, but gas-permeable and is, for example, made from a material which can be obtained under the trade name “Tyvek”. The function of the lid 8 is described below with the aid of FIG. 2.

First the sterilization chamber 4 is evacuated by means of a suitable vacuum pump 9. The sterilization chamber 4 is subsequently isolated from the vacuum pump 9 by means of closing of a valve 10, so that the suction action is halted.

To ensure that the overheated hydrogen peroxide vapor located in the evaporator 1 reaches the sterilization chamber 4 via the conduit 12 in flow direction B, a valve 11 is opened. The pressure in the evaporator 1 must as a consequence be higher than the pressure in the sterilization chamber 4. During expansion, the volume of the hydrogen peroxide vapor increases, which results in the vapor cooling down well below the dew point, and condensating on all surfaces of the objects 7 and the packages 6 as well as the support surface 5 and the inner surfaces of the sterilization chamber 4. After the desired duration of the sterilization treatment, the condensate can be drawn off with the aid of the vacuum pump 9 and the sterilization chamber 4 can be filled via a conduit 13 and a valve 14 with a sterile gas.

FIG. 2 shows, greatly enlarged in relation to FIG. 1, an example of an embodiment according to the present invention of a multi-packaging 6, in the case of which the sterilization of syringes 15 made of glass or plastic having injecting needles 16 adhered thereto is involved, said syringes 15 hanging by means of a collar 17 in a holed plate 18 in large numbers, for example 100 to 200 syringes, in a tub 19 made of plastic. The edge 20 of the holed plate 18 lies hereby on a shoulder 21 of the tub 19, which is deep enough to prevent the injecting needles 16 from touching the tub bottom 22.

The tub 19 is open at the top 23 and is covered with a gas-permeable, but bacteria-impermeable material (for example Tyvek). This cover 24 is shaped like a sheet and resembles a heavy paper, but is significantly more tear-resistant. The perforations, which arise, for example, from the weaving of suitable threads, are so fine that the overheated hydrogen peroxide vapor can penetrate through the cover 24, which cover 24, however, prevents microorganisms from reaching the inside. The cover 24 is sealed to the tub 19 by means of a sealing seam 26.

The sealed tub 19 is in turn packed in a bag 25, which is itself partly or completely made of the same or similar material as the cover 24 and which provides the tub 19 with a second gas-permeable, but bacteria-impermeable seal. The bag 25 is made of two parts sealed together along a sealing seam 27.

After the syringes 15 have been placed in the tub 19, and the tub 19 sealed with the cover 24, and the tub 29 then placed in the bag 25, everything in the sterilization chamber 4, including the other packages 6, is sterilized in one operation. After they have been removed from the sterilization chamber 4, the syringes 15, including the injecting needles 16, as well as the entire tub 19 are sterile. This applies also to the intermediary space between the bag 25 and the tub 19.

The packaging shown in FIG. 2 avoids a recontamination of the tub 19 at the filling installation, where it is removed from the bag 25 and placed via a lock chamber in a sterile clean room.

For lesser requirements, or if the sterilization process is to take place only at the filling installation, the bag 25, as additional packaging can, of course, be omitted.

1. Process for sterilizing objects, in particular medical objects, which are packed in a non-sterile state into a bacteria-impermeable, but gas-permeable non-sterile package and subsequently, for the purposes of chemical sterilization, covered by a reactive gas-like medium which penetrates into the package as a result of a difference in pressure, and which is replaced by a sterile gas after the sterilization process, wherein the medium is overheated hydrogen peroxide vapor, which is forced below the dew point in the inside of the packaging and thus forced to condensate, and wherein the condensate is returned to the vapor phase by means of evacuation of the packaging and thereafter pumped out.

2. A process according to claim 1, wherein the overheated hydrogen peroxide vapor is fed from an evaporator to a sterilization chamber, which holds up to at least one package.

3. A process according to claim 1 or 2, wherein the condensation process and the subsequent evacuation are repeated at least once.

4. A process according to claim 1 or 2, wherein the condensation process is repeated at least once without an intermediary evacuation process.

5. A process according to any one of the claims 1 to 4, wherein, in order to accelerate or reinforce the condensation process, the package is flooded with a sterile gas.
